(19) United States
(12) Patent Application Publication
(10) Pub. No.: US 2018/0275027 A1
(43) Pub. Date: Sep. 27, 2018

(54) CELL YIELD FOR SYNTHETIC TISSUE CONTROLS AND SYNTHETIC TISSUE MICROARRAY CONTROLS

(71) Applicant: SLMP, LLC, McKinney, TX (US)

(72) Inventors: Syed Ashraf IMAM, North Hollywood, CA (US); Mark Lee REES, Huntington, NY (US)

(21) Appl. No.: 15/466,421

(22) Filed: Mar. 22, 2017

(51) Int. Cl. G01N 1/36; G01N 33/96; C12N 5/071; C12N 5/09; G01N 33/574; C12Q 1/68

(52) U.S. Cl. CPC G01N 1/36; G01N 33/96; C12N 5/0697; G01N 2001/368; G01N 33/574; C12Q 1/6886; C12N 5/0693

(57) ABSTRACT

The disclosed embodiments include methods to improve yield of cultured cells of STC and STMC for use in determining presence of cancer, and methods to detect presence of cancer. In one embodiment, the method includes solidifying a fluidly gel to form a solid cell holder. The method also includes forming a cavity in the cell holder to hold co-cultured cells, the cell holder operable to hold a plurality of cell culture bags of co-cultured cells, the co-cultured cells comprising normal cells and cancer cells of a type of cancer. The method further includes depositing co-cultured cells from at least two of the plurality of cell culture bags of co-cultured cells in the cavity of the cell holder and processing the co-cultured cells to form the synthetic tissue control.

### BACKGROUND

Pathology testing and clinical laboratory testing are important aspects of modern diagnostic and prognostic practices. Control samples are often used to maintain quality control (QC) for reproducibility of test results by immunohistochemical (IHC) staining, in situ hybridization (ISH), and other methods of molecular analyses.

Some of the controls available for IHC staining and ISH staining of tumor tissues and other diseased tissues are cancer tissue-derived controls. However such types of controls are only available in very limited quantities, and once such controls are exhausted, replacement controls with the same characteristics may be unavailable. Other types of available controls are cancer cell lines-derived controls. However, such types of controls do not exhibit consistent patterns and levels of cellular expression of a given marker, or heterogeneity of said expression, which is ubiquitous to tumor tissues. As such, these controls have little or no morphological resemblance to actual tumor tissues. Further, conventional techniques to form controls involve processes that produce poor yield, thereby requiring significant amounts of cultured cells in order to form such controls, thereby increasing productivity costs and decreasing productivity efficiency.

### BRIEF SUMMARY OF THE DISCLOSED EMBODIMENTS

The disclosed embodiments provide methods for forming synthetic tissue controls and synthetic tissue microarray controls for IHC and ISH tests for cancer diagnosis and prognosis, as well as methods for determining presence of one or more types of cancer.

In accordance with an illustrative embodiment, a method for determining presence of at least one type of cancer is provided. The method includes staining a portion of a synthetic tissue control (STC). The STC includes normal cells and cancer cells of a type of cancer co-cultured based on at least one cell culturing factors. The at least one co-culture factors includes one or more of the following factors: A type of the cancer cell being cultured, a ratio of the cancer cells to the normal cells being co-cultured, seeding density of the cells being cultured, a type of cell growth supplement used to facilitate co-culturing the cells, and a concentration of the cell growth supplement used to facilitate co-culturing the cells. The method further includes observing the stained portion of the STC to determine a presence of one or more biomarker types, the one or more biomarker types indicating presence of the cancer cells.

In accordance with an illustrative embodiment, a method to form a synthetic tissue control for use in determining presence of cancer is provided. The method includes culturing cells comprising normal cells and cancer cells of a type of cancer based on at least one cell culturing factors. The at least one cell culturing factors includes a type the cancer cells being cultured, a ratio of the cancer cells to the normal cells being cultured, and seeding density of the cells being cultured.

In accordance with another illustrative embodiment, a synthetic tissue microarray is provided. The synthetic tissue microarray includes a plurality of STCs, each STC of the plurality of STCs includes normal cells and cancer cells of a type of cancer. The normal cells and the cancer cells are cultured based on at least one cell culturing factors. The at least one cell culturing factors includes a type of the cancer cells being cultured, a ratio of the cancer cells to the normal cells being cultured, seeding density of the normal cells and cancer cells being cultured, and a type of cell growth supplement used to facilitate culturing and growth of the normal cells and cancer cells.

Additional details of the disclosed embodiments are provided below in the detailed description and corresponding drawings.

### BRIEF DESCRIPTION OF THE DRAWINGS

Illustrative embodiments of the present invention are described in detail below with reference to the attached figures, which are incorporated by reference herein, and wherein:

FIG. 1 is an illustration of a synthetic tissue microarray that includes four controls in accordance with one embodiment.

FIG. 2A illustrates a side view of a fluidly gel being solidified to form a cell holder in accordance with one embodiment.

FIG. 2B illustrates a side view of the cell holder of FIG. 2A having a cavity in the cell holder in accordance with one embodiment.

FIG. 2C illustrates a top down view of the cell holder of FIG. 2A in accordance with one embodiment.

FIG. 2D illustrates another top down view of the cell holder of FIG. 2A having a dye applied to co-cultured cells deposited into the cavity in accordance with one embodiment.

FIG. 3A illustrates an image of synthetic tissue containing breast cancer cells, which has been stained with a specific antibody to HER-2/nue to illustrate presence of HER-2/nue expression in accordance with one embodiment.

FIG. 3B illustrates an image of breast tumor tissue, which has been stained with a specific antibody to HER-2/nue to illustrate presence of HER-2/nue expression in accordance with one embodiment.

FIG. 3C illustrates an image of the synthetic tissue of FIG. 3A which has been stained with pre-absorbed antibody HER-2/nue as a test of specificity of the stain in accordance with one embodiment.

FIG. 3D illustrates an image of the breast tumor tissue of FIG. 3B which has been stained with pre-absorbed antibody HER-2/nue as a test of specificity of the stain in accordance with one embodiment.

FIG. 4A illustrates an image of synthetic tissue stained to illustrate presence of E Cadherin marker in accordance with one embodiment.

FIG. 4B illustrates an image of breast tumor tissue stained to illustrate presence of E Cadherin marker in accordance with one embodiment.

FIG. 4C illustrates an image of synthetic tissue stained to illustrate presence of Estrogen Receptor marker in accordance with one embodiment.

FIG. 4D illustrates an image of breast tumor tissue stained to illustrate presence of Estrogen Receptor marker in accordance with one embodiment.

FIG. 4E illustrates an image of synthetic tissue stained to illustrate presence of a cell proliferation (Ki-67) marker in accordance with one embodiment.

FIG. 4F illustrates an image of breast tumor tissue stained to illustrate presence of a cell proliferation (Ki-67) marker in accordance with one embodiment.

FIG. 5 illustrates examples of antibodies for detecting different types of cancers that are covered by the synthetic tissue controls and the synthetic tissue microarrays described herein, in accordance with one embodiment.

The illustrated figures are only exemplary and are not intended to assert or imply any limitation with regard to the environment, architecture, design, or process in which different embodiments may be implemented.

### DETAILED DESCRIPTION

A 3-D Synthetic tissue control (STC) is generated by co-culturing normal cells and a type of cancer cells under defined and controlled conditions in suspension. As defined herein, “normal” cells include any non-tumor cells. Normal cells may be formed from stromal cells and other suitable cell types.

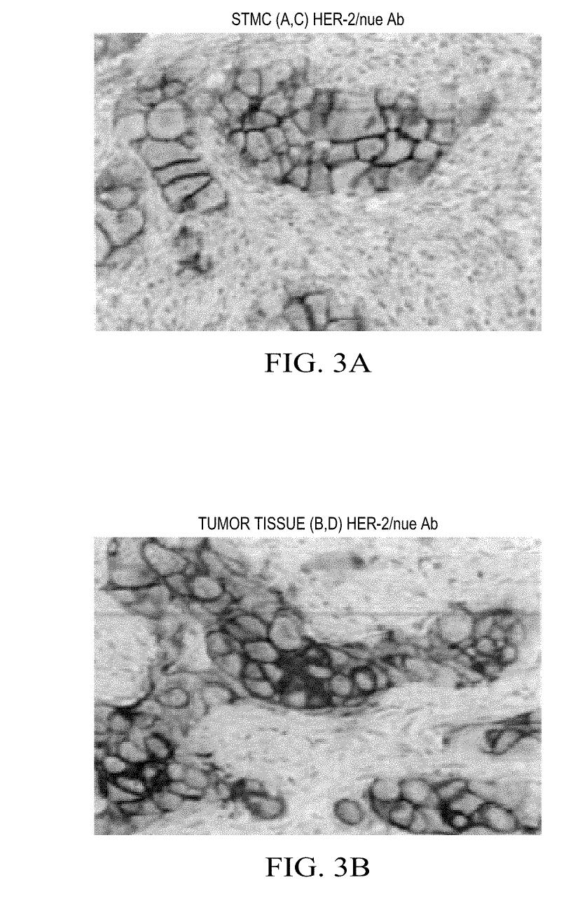

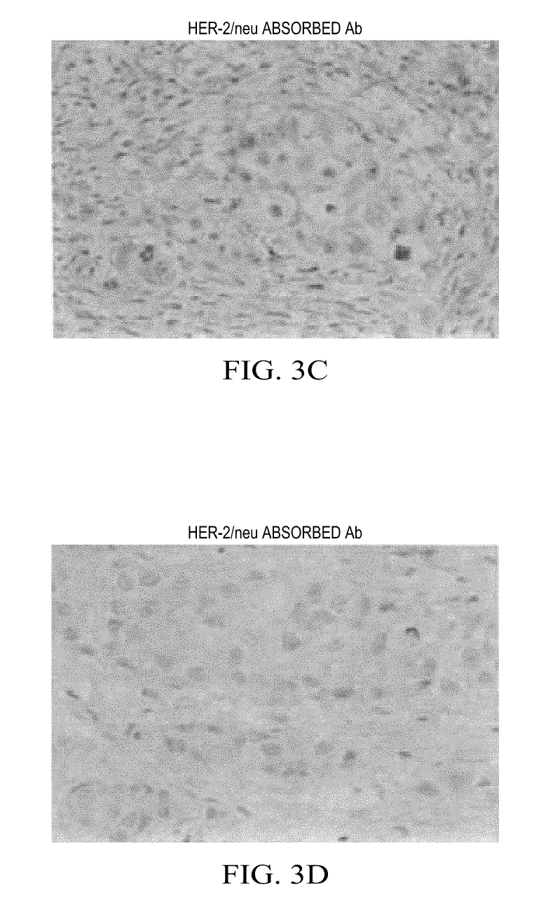

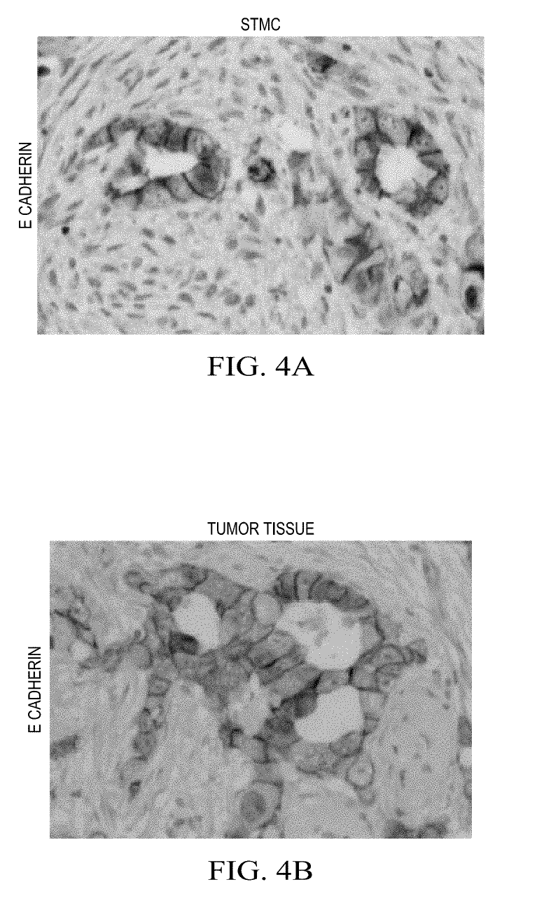

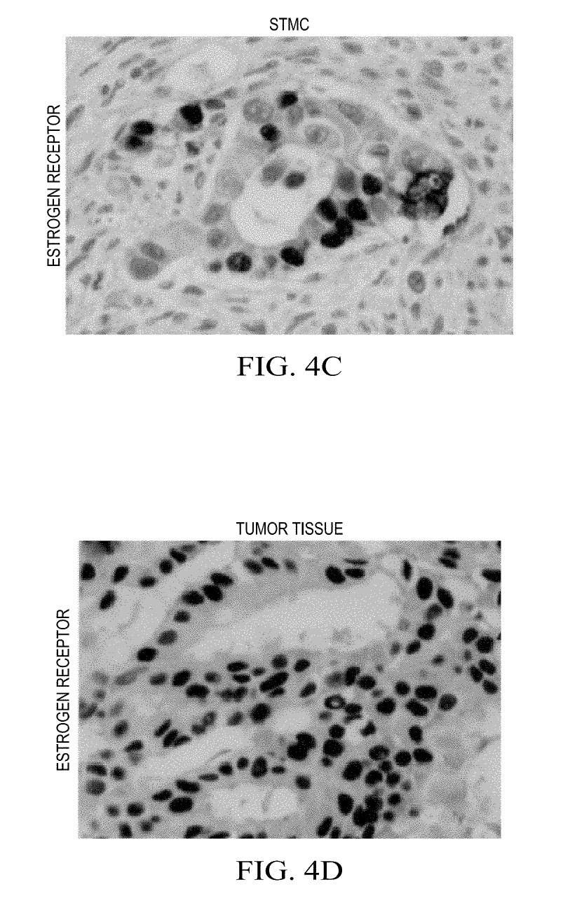

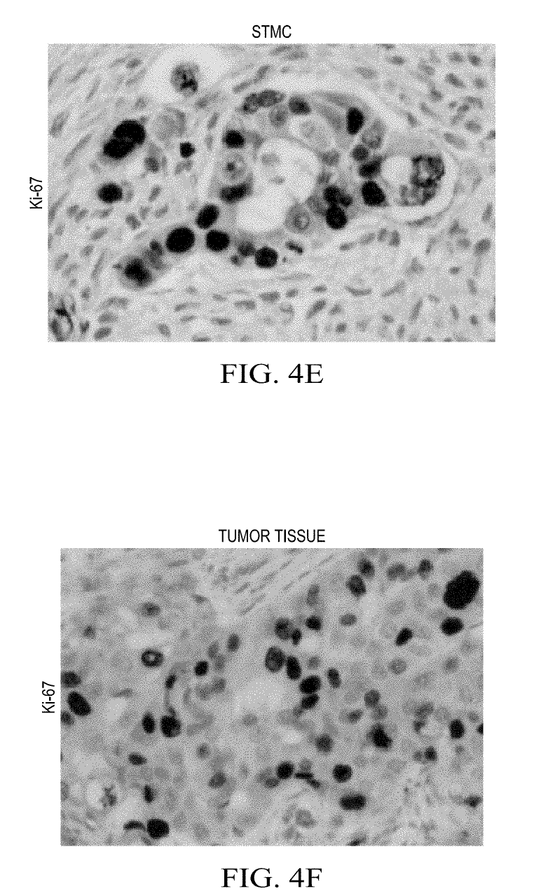

The STC reproducibly exhibits expected patterns and levels of tumor tissue associated cellular and extracellular (ECM) markers and architecture with close resemblance to tumor tissue. Examples of the close resemblance between STCs and tumor tissues are illustrated in FIGS. 3A-3D and FIGS. 4A-4E. 3-D Synthetic tissue microarray controls (STMC) are constructed from multiple STCs. In a preferred embodiment, STC and STMC are prepared as formalin-fixed and paraffin-embedded (FFPE) blocks or precut into sections for various markers used in pathology testing laboratories. Examples of compatible markers include various markers discussed in detail in the below description, as well as illustrated in the figures. The STC and STMC blocks and sections can be used as both positive and negative controls for IHC staining and ISH staining of tumor tissue and other diseased tissues. The processes for forming STCs and STMCs and using the STCs and STMCs to detect presence of cancer are provided in detail in the paragraphs below.

Cell Culture of STC and STMC

STCs are cultured in an approximately zero gravity culture, usually in the form of a formalin-fixed and paraffin-embedded (FFPE) cell block, where each STC contains a type of cancer cells and stromal cells. In some embodiments, cancer cells and normal stromal cells are separately cultured in cell-culture flasks. In one of such embodiments, a DNase of approximately 5 microgram per milliliter of cell culture medium is added to each cell-culture flask that contains either cancer cells or normal stromal cells. The addition of DNase prevents clumping of the harvested cancer cells or normal stromal cells from the cell-culture flasks. As a result, cell counts of the cancer cells and/or the normal stromal cells may be accurately determined and un-clumped cancer cells and normal stromal cells may be harvested.

In some embodiments, two or more cell-types (i.e., cancer cells and normal stromal cells) are co-cultured under stringently defined conditions and within a controlled environment. In some embodiments, the cancer cells and the stromal cells are co-cultured in a plurality of cell culture bags of cells. As defined herein, a cell culture bag is a container having a built-in port for adding and removing cells. In some embodiments, the cell culture bag is sterile. In further embodiments, the build-in port of the cell culture bag includes an air tight cap. The bag holds cancer and stromal cells during the co-culture stage of production. In some embodiments, approximately 80 million stromal cells and/or cancer cells may be harvested from each cell culture bag after approximately ten days of co-culturing. In other embodiments, approximately 30-120 million stromal cells and/or cancer cells may be harvested from each cell culture bag after approximately two weeks of co-culturing. The dimensions of the cell culture bags may vary based on the type of cells being co-cultured as well as the type of nutrients optimal for co-culturing such cells. In one of such embodiments, a DNase of approximately 5 microgram per milliliter of cell culture medium is added to each cell culture bag containing cancer cells and normal stromal cells, where the cell culture bag is mounted on a bioreactor in a CO₂-incubator during the co-culturing process. The addition of the DNase facilitates cells to remain in an un-clumped condition in suspension during co-culture. More particularly, the addition of DNase facilitates the normal stromal cells to remain un-clumped, and thus allows the normal stromal cells to form a homogenous core. Further, the addition of DNase facilitates the cancer cells to remain un-clumped, and thus facilitates un-clumped cancer cells to invade the homogenous core. In another one of such embodiments, a Fibronectin of approximately 1 microgram per milliliter of the cell culture medium is added on the first day to co-culture in each cell culture bag containing the cancer cells and the normal stromal cells. In such embodiment, the addition of Fibronectin facilitates formation of basement membrane-like structures during an early phase of growth of the co-culture cells. Further, the addition of the Fibronectin facilitates an improved contact between the co-cultured cancer cells and the normal stromal cells. The formation of the basement membrane-like structures facilitates the co-cultured cells to resemble actual tumor tissue.

In a preferred embodiment, the cancer cells and the stromal cells are co-cultured in an approximately zero gravity environment for a period of eight to twelve days. Furthermore, the cancer cells and the stromal cells are co-cultured in a cell culture chamber configured to maintain a concentration of CO₂ and temperature inside and outside the chamber at levels based on at least one cell culturing factors to allow the cells to develop characteristics that are similar to or identical to actual tumor tissue. A motorized rotating device holds and slowly spins the cell culture chambers at a speed that is based on the at least one cell culturing factors to allow the cells to develop characteristics similar to or identical to characteristics of actual tumor tissue.

Cell culturing factors include, but are not limited to, the type of cancer cells being cultured, the ratio of the cancer cells to the stromal cells, seeding density of the cells being cultured, and concentration and type of cell growth supplement used to facilitate culturing the cells to develop characteristics that are similar to or identical to actual tumor tissue. In one example embodiment, a ratio of the cancer cells for a breast cancer cell line (MCF.7) to the normal stromal cell (fibroblast) is 1 in 99.8, respectively, to produce the STC. Additionally, 10 micrograms of insulin are used as a growth factor supplement to facilitate growth of the MCF 7. Furthermore, the seeding density of MCF.7 cancer cells is 18,750 per milliliter, whereas that of the fibroblast 187,876 per milliliter. Similarly, STMCs are also cultured based on the above described process. In one embodiment, STC and STMC are cultured based on one of the above-identified factors. In another embodiment, STC and STMC are cultured based on two of the above-identified factors. In a further embodiment, STC and STMC are cultured based on three of the above-identified factors. In a further embodiment, STC and STMC are cultured based on all of the above-identified factors.

STC and STMC are cultured, based on at least one of the previously stated cell culturing factors, to provide control ‘faux’ tissues having known patterns and levels of expression of various markers, including proteins, RNAs, DNAs and other components of interest, for diagnosis, prognosis, and selection of patients for a particular specific/targeted therapy. STC and STMC may be cultured for standard expression of markers employed in IHC as diagnostic and predictive markers for treatment response, exemplified by tests for Epidermal Growth Factor Receptor-2 (HER-2/nue), estrogen receptor (ER), progesterone receptor (PR), Ki-67, and other types of suitable diagnostic and predictive markers for treatment response. STC and STMC may further be cultured for standard expression of markers that are employed in IHC as predictive markers to treatment response, exemplified by tests for HER-2/nue, Met 4, as well as other types of suitable predictive markers for treatment response. Furthermore, STC and STMC may also be cultured or standard expression of markers employed in immunofluorescence techniques.

STC and STMC may also be cultured to provide a consistent level of expression when observed via a fluorescence in situ hybridization (FISH) based technique for RNA and/or DNA markers. In certain embodiments, the expression may include expression levels of RNA translocations. In other embodiments, the expression may include expression levels of DNA mutations. STC and STMC may also be cultured to provide a consistent level of expression when observed via a chromogenic in situ hybridization (CISH) based technique for RNA and/or DNA markers. In certain embodiments, the expression may include expression levels of RNA translocations and/or DNA mutations. As such, an almost limitless range of biomarkers may be provided by selection of cell lines that are employed to make the STC or STMC.

STC and STMC may be cultured, based on at least one of the previously stated cell culturing factors, to consistently provide various levels of expressions of biomarkers used in IHC and ISH staining. In some embodiments, STC and STMC are cultured to provide high expression (HE or 3+) of biomarkers used in IHC and ISH staining. In other embodiments, STC and STMC are cultured to have medium expression (ME or 2+) of biomarkers used in IHC and ISH staining. In further embodiments, STC and STMC are cultured to have low expression (LE or 1+) of biomarkers used in IHC and ISH staining. In some embodiments, co-cultured cells from the plurality of cell culture bags are deposited into a centrifuge tube to mix the co-cultured cells and to combine the co-cultured cells for processing and embedding. As described here. The co-cultured cells product is tightly wrapped around in a biopsy filter paper and placed in a tissue processing equipment, such as the centrifuge or another processing equipment described herein.

Processing and Embedding STC and STMC

The cultured STC is processed and then embedded. A cell holding device having a cavity utilized to hold co-cultured cells from at least two cell culture bags is utilized to hold the co-cultured cells during the processing and the embedding of the co-cultured cells. In some embodiments, the cell holding device is formed from a fluidly gel that solidifies at room temperature. In some embodiments, the fluidly gel is placed in a reservoir having a shape that is similar to a desired shape of the fluidly gel once the fluidly gel solidifies. An object is inserted into the fluidly gel during the solidification of the fluidly gel and subsequently removed from the fluidly gel once the fluidly gel solidifies to form the cavity. The size and shape of the cavity are defined by an external shape and size of the object. In some embodiments, different objects having different external sizes and shapes are utilized to form cavities having different sizes and shapes based on a desired amount of co-cultured cells to be processed, the type of the cancer cells to be processed, the quantity of desired STC, as well as other factors described herein. The cavity securely holds the co-cultured cells to prevent the co-cultured cells from dissipating into the surrounding medium during the processing and embedding processes described herein.

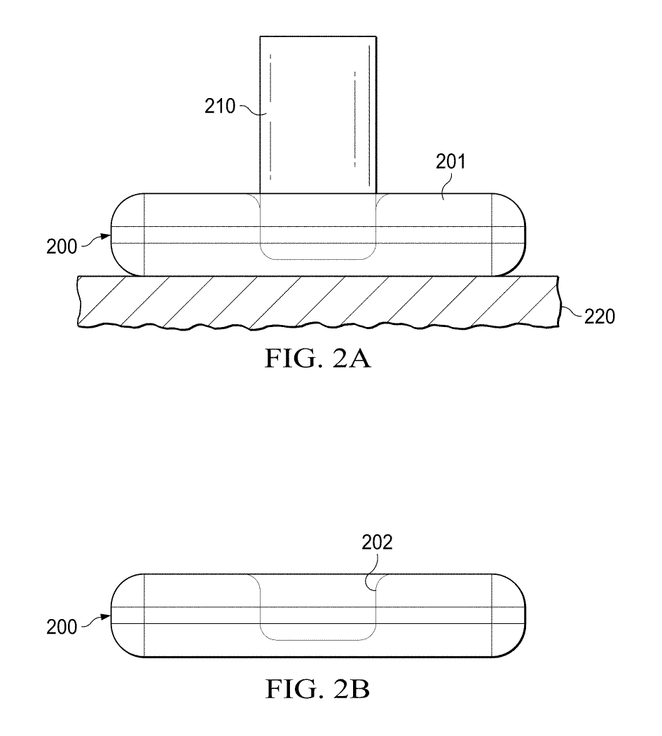

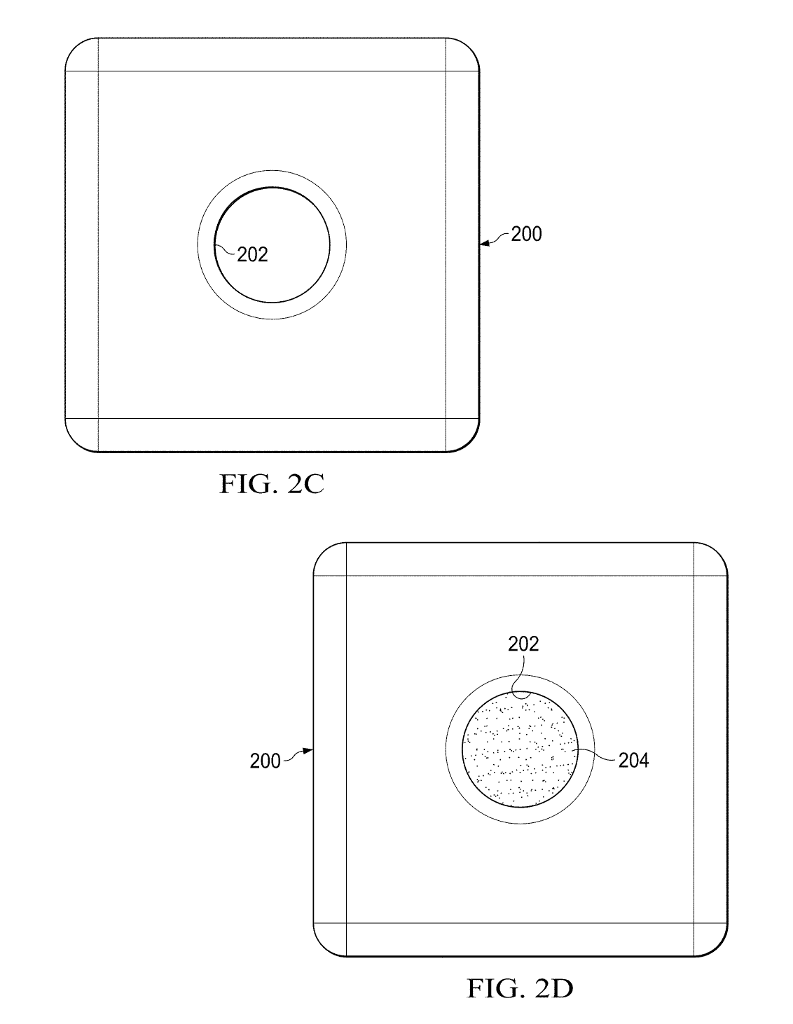

As described herein, the cavity is operable to hold co-culture cells harvested from two cell culture bags. In some embodiments, the cavity is operable to store the co-cultured cells from additional cell culture bags, thereby further increasing the yield of desirable cell cores. In some embodiments, when a harvested co-cultured cell product is transferred into the cavity of the cell holding device, a colored dye is added to the harvested co-cultured cell product. More particularly, a dye having a pre-determined color is added to the harvested co-cultured cell product without touching such product, thereby allowing the dye to remain confined within the cavity. The addition of a colored dye to the harvested co-cultured cell product prior to the harvested co-cultured cell product is embedded in paraffin facilitates an accurate means to identify the harvested co-cultured cell product. In some embodiments, a filter material, such as a biopsy filter paper is then wrapped around the cell holding device containing the harvested co-cultured cell product and the dye within its cavity. The cell holding device is then placed in a histological cassette and transferred onto a tissue processing tray. Additional processes, such as vacuum infiltration process and paraffin embedding process may then be performed on the harvested co-cultured cell product as a block. In some embodiments, the harvested co-cultured cell product is fixed in formalin for subsequent processing and embedding. In other embodiments, the harvested co-cultured cell product is fixed in bousin or another fixative solution for subsequent the processing and embedding. As described herein, the cavity is operable to hold co-cultured cells harvested from at least two cell culture bags. Additional descriptions of the cell holding device, forming the cell holding device, and depositing co-cultured cells into the cavity of the cell holding device are provided in the paragraphs below and are illustrated in at least FIGS. 2A-2D.

In one embodiment, the diameter of the cultured STC is approximately 0.04 Cm. In another embodiment, the diameter of the cultured STC may be within a range of 0.01-0.04 Cm. Contrary to the STC, the tissue specimen may have a diameter of 2-4 Cm. Given the size of the cultured STC, a processing and an embedding device having a mesh with pore size approximately 0.001 cm is used to hold the tissue specimen during the embedding process. In another embodiment, the pore size of the embedding device is within a range of 0.001-0.004 Cm. A plurality of cultured STCs that provide a desired level of expression of desired biomarkers is embedded to form a STMC.

Staining STC and STMC

A section from each block of the embedded STC is evaluated by IHC staining techniques to identify individual constructs. In some embodiments, the individual constructs in the embedded STC represent 50 to 60% from the total of approximately 500 individual constructs with the desired combination of cancer cells and normal stromal cells and the invasion of normal stromal cell core by the cancer cells. In other embodiments, the individual constructs in the embedded STC represent 80% from the total of 500-600 individual constructs with the desired combination of cancer cells and normal stromal cells and the invasion of normal stromal cell core by the cancer cells. In some embodiments, only constructs with characteristics that are similar or identical to actual tumor tissue are selected as STC.

Individual constructs from each block containing a certain combination of co-cultured cell types with characteristics that are similar or identical to actual tumor tissue are mechanically removed from the original block and are used to construct a STMC. The STMC is constructed to include a plurality of types of cancer cells with varying levels and patterns of expression of markers of interest. The STC and STMC may be viewed by laboratory operators via a variety of devices such as microscopes, whole slide imaging (WSI) devices, and other suitable devices for observing expressions of biomarkers.

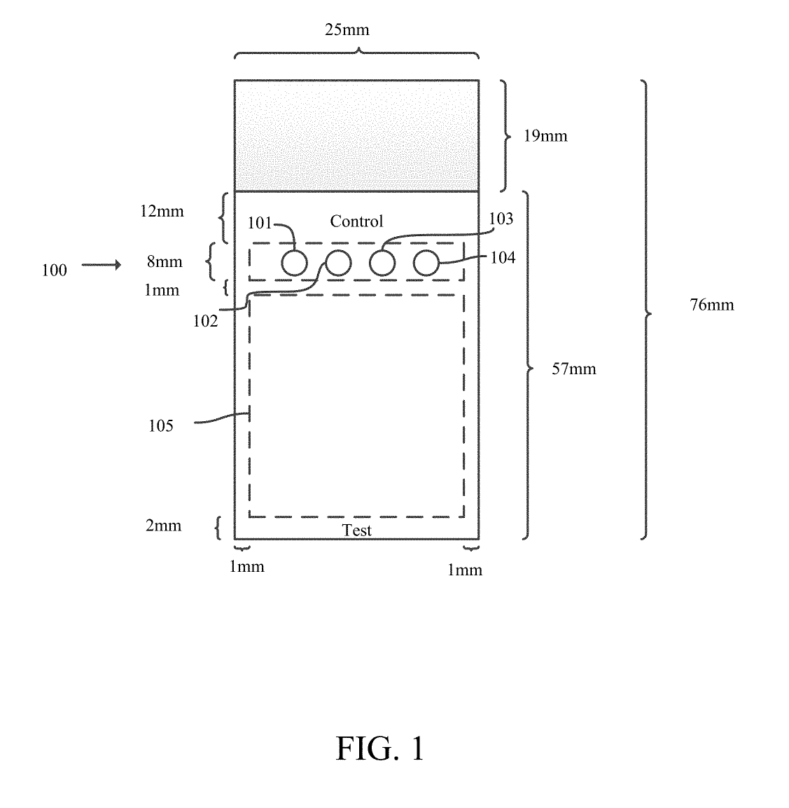

FIG. 1 is an illustration of a synthetic tissue microarray 100 that includes four STCs 101, 102, 103, and 104 in accordance with one embodiment. In the embodiment illustrated in FIG. 1, controls 101, 102, 103, and 104 are placed proximate to test tissue 105 on the same histologic slide. The controls 101, 102, 103, and 104 and the test tissue 105 are stained with one or more stains for different types of biomarkers.

In some embodiments, the synthetic tissue microarray 100 is stained to observe for expression of markers employed in IHC as diagnostic markers and predictive markers for treatment response. In other embodiments, the synthetic tissue microarray 100 is stained to observe for expression of markers employed in IHC as predictive markers for treatment response. In further embodiments, the synthetic tissue microarray 100 is stained and observed via a FISH technique, for a level of expression of RNA and DNA markers. In further embodiments, the synthetic tissue microarray 100 is stained and observed via a CISH technique, for a level of expression of RNA and DNA markers.

In some embodiments, the synthetic tissue microarray 100 provides positive controls for at least one type of cancer. In other embodiments, some controls of the synthetic tissue microarray 100 provide positive controls while other controls of the synthetic tissue microarray 100 provide negative controls. The synthetic tissue microarray 100 may be cultured to provide high expression, medium expression, or low expression of the markers. Although the embodiment illustrated in FIG. 1 includes four controls 101, 102, 103, and 104, the synthetic tissue microarray 100 may be formed from a different number of controls. A laboratory operator may examine the synthetic tissue microarray 100 under a variety of devices such as microscopes and WSI devices to compare the stained controls with the stained test tissue to determine presence or absence of expression of markers in test tumor tissues.

FIG. 2A illustrates a side view of a fluidly gel 201 being solidified to form a cell holder 200 in accordance with one embodiment. The fluidly gel 201 is deposited proximate a cooling object 220 to cool the temperature of the fluidly gel 201, thereby solidifying the fluidly gel 201 to form the cell holder 200. In some embodiments, the fluidly gel 201 is a solid at room temperature and is initially heated up such that the fluidly gel 201 is in a fluid state. In some embodiments, the fluidly gel 201 is placed in a reservoir during the solidification process to take on a shape approximately defined by an internal surface of the reservoir. In some embodiments, the cooling object 220 is ice that facilities cooling of the fluidly gel 201. An object 210 is inserted into the fluidly gel 201 while the fluidly gel 201 is solidifying and is removed from the fluidly gel 201 after the fluidly gel 201 has solidified to form a cavity (shown in FIGS. 2B-2D) of the cell holder 200. In some embodiments, the object 201 is a tube having a shape and dimensions proximate a desired shape and dimensions of the cavity. As stated herein, the cavity is operable to hold co-cultured cells from at least two cell culture bags described herein.

FIG. 2B illustrates a side view of the cell holder 200 of FIG. 2A having a cavity 202 in the cell holder 200 in accordance with one embodiment. FIG. 2C illustrates another image of the cell holder 200 of FIG. 2A having the cavity 202 in accordance with one embodiment. Although FIGS. 2B and 2C illustrate one cavity 202 formed on the cell holder 200, multiple cavities, each operable to hold co-cultured cells from at least two cell culture bags may be formed on the cell holder 200. Further, although the cavity of FIG. 2C punctures through opposite surfaces of the cell holder 200, other cavities may not puncture through the cell holder 200. As described herein, the cavity 202 may take on a variety of shapes and dimensions, including the shape illustrated in FIGS. 2B and 2C.

FIG. 2D illustrates another top down view of the cell holder 200 of FIG. 2A having a dye applied to co-cultured cells 204 deposited into the cavity in accordance with one embodiment. In the embodiment of FIG. 2D, a blue colored dye is applied to the co-cultured cells 204 that are deposited in the cavity 202. The amount of dye applied may be based on a variety of factors including the number of co-cultured cells deposited in the cavity 202, the type of cancer cells deposited in the cavity 202, the size and dimensions of the cavity 202 as well as other factors described herein. In some embodiments, a pre-selected colored dye is carefully added to the co-cultured cells product within the cavity 202 to ensure that the colored dye remains confined within the cavity 202. Although FIG. 2D illustrates a blue dye applied in the cavity 202, a different colored dye may also be applied to facilitate identification of the co-cultured cells. In some embodiments, a filter material, such as filter paper is applied over the cavity. In some embodiments, the cell holder 200 is then transferred onto a pathology cassette (not shown) and the pathology cassette is processed to form STC. In some embodiments, the STC is then embedded on a paraffin block to form a STMA.

FIG. 3A illustrates an image of synthetic tissue containing breast cancer cells in accordance with one embodiment. FIG. 3B illustrates an image of breast tumor tissue in accordance with one embodiment. The synthetic tissue illustrated in FIG. 3A has been cultured under conditions described herein. As shown in FIGS. 3A and 3B, the synthetic tissue containing breast cancer cells and the actual breast tumor tissues exhibit significantly similar characteristics.

FIG. 3C illustrates an image of the synthetic tissue of FIG. 3A after the synthetic tissue is stained with pre-absorbed antibody to HER-2/nue to illustrate a specificity of the stain for HER-2/nue marker in accordance with one embodiment. FIG. 3D illustrates an image of the breast tumor tissue of FIG. 3B after the tumor tissue is stained with pre-absorbed antibody to HER-2/nue to illustrate a specificity of the stain for HER-2/nue marker in accordance with one embodiment. As shown in FIGS. 3C and 3D, the stained synthetic tissue and the stained tumor tissue exhibit significantly similar characteristics, which allow the synthetic tissue to be used as a control for standard expression of markers that are employed in IHC as a diagnostic marker or as a predictive marker for treatment response such as HER-2/nue. Other examples of expression of markers that are employed in IHC as predictive markers for treatment response include Met 4, as well as other suitable diagnostic markers or predictive markers for treatment response. In further embodiments, the synthetic tissues illustrated in FIGS. 3A and 3C may also provide a level of expression of RNA and DNA markers when observed via a FISH technique. In further embodiments, the synthetic tissues illustrated in FIGS. 3A and 3C may also provide a level of expression of RNA and DNA markers when observed via a CISH technique.

FIG. 4A illustrates an image of synthetic tissue stained to illustrate presence of E Cadherin marker in accordance with one embodiment. FIG. 4B illustrates an image of breast tumor tissue stained to illustrate presence of E Cadherin marker in accordance with one embodiment. FIG. 4C illustrates an image of synthetic tissue stained to illustrate presence of Estrogen Receptor marker in accordance with one embodiment. FIG. 4D illustrates an image of breast tumor tissue stained to illustrate presence of Estrogen Receptor marker in accordance with one embodiment. FIG. 4E illustrates an image of synthetic tissue stained to illustrate presence of a proliferation (Ki-67) marker in accordance with one embodiment. FIG. 4F illustrates an image of breast tumor tissue stained to illustrate presence of a proliferation (Ki-67) marker in accordance with one embodiment.

The synthetic tissues illustrated in FIGS. 4A, 4C, and 4E have been cultured under conditions described herein. As shown in FIGS. 4A-4F, the synthetic tissues and the stained tumor tissues exhibit significantly similar characteristics, which allow the synthetic tissues to be employed in IHC as diagnostic markers and predictive markers for treatment response, exemplified by tests for E Cadherin, ER, progesterone receptor, and Ki-67, as well as tests for other suitable types of diagnostic markers and predictive markers for treatment response. In other embodiments, the synthetic tissues illustrated in FIGS. 4A, 4C, and 4E may also be used to provide for expressions of biomarkers employed in IHC as predictive markers for treatment response. In further embodiments, the synthetic tissues illustrated in FIGS. 4A, 4C, and 4E may also provide a level of expression of RNA and DNA markers when observed via a FISH technique. In further embodiments, the synthetic tissues illustrated in FIGS. 4A, 4C, and 4E may also provide a level of expression of RNA and DNA markers when observed via a CISH technique.

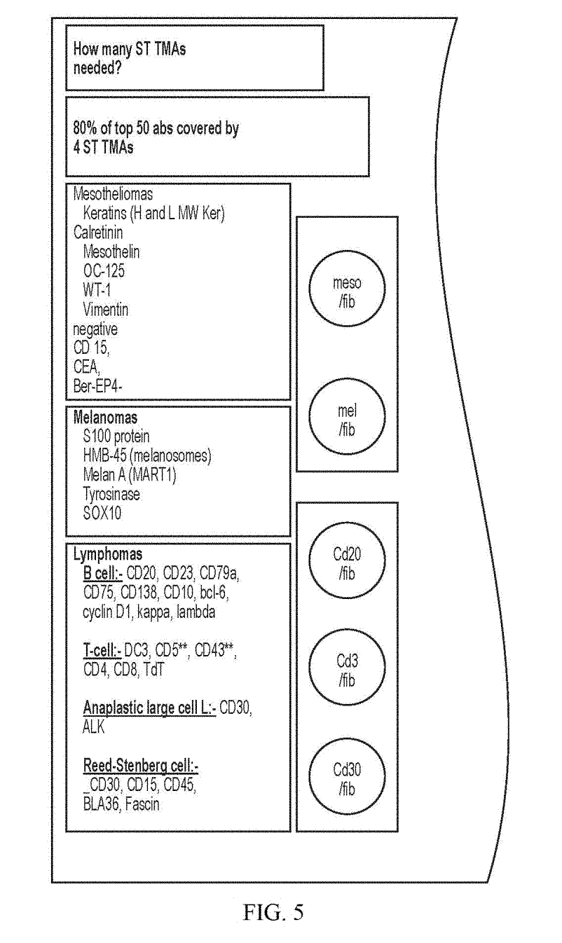

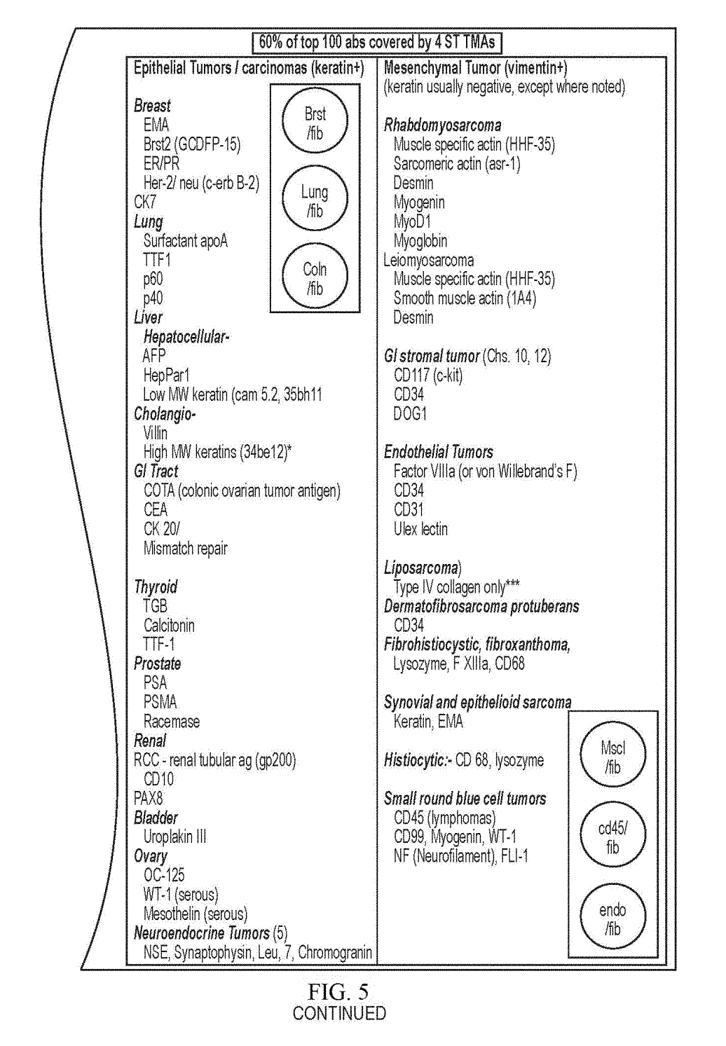

FIG. 5 illustrates examples of antibodies for detecting different types of cancers that are covered by the synthetic tissue controls and the synthetic tissue microarray controls described herein, in accordance with one embodiment. As illustrated in FIG. 5, STC and STMC can be used to test a variety of types of cancers including, but not limited to breast cancer, lung cancer, liver cancer, thyroid cancer, prostate cancer, colon cancer, cervical cancer, kidney cancer, ovarian cancer, melanoma cancer, brain cancer, leukemia, lymphomas as well as other types of cancers.

The above disclosed embodiments have been presented for purposes of illustration and to enable one of ordinary skill in the art to practice the disclosed embodiments, but is not intended to be exhaustive or limited to the forms disclosed. Many insubstantial modifications and variations will be apparent to those of ordinary skill in the art without departing from the scope and spirit of the disclosure. For instance, although the flowcharts depict a serial process, some of the steps/blocks may be performed in parallel or out of sequence, or combined into a single step/block. The scope of the claims is intended to broadly cover the disclosed embodiments and any such modification. Further, the following clauses represent additional embodiments of the disclosure and should be considered within the scope of the disclosure:

Clause 1, a method to increase yield of co-cultured cells of a synthetic tissue control, the method comprising solidifying a fluidly gel form a solid cell holder; forming a cavity in the cell holder to hold co-cultured cells, the cavity being operable to hold a plurality of cell culture bags of co-cultured cells, the co-cultured cells comprising normal cells and cancer cells of a type of cancer co-cultured based on at least one cell culturing factors, the at least one co-culture factors comprising a type of the cancer cells being co-cultured, a ratio of the cancer cells to the normal cells being co-cultured, seeding density of the normal cells and the cancer cells being co-cultured, a type of cell growth supplement used to facilitate co-culturing the normal cells and the cancer cells, and a concentration of the cell growth supplement used to facilitate co-culturing the normal cells and the cancer cells; depositing co-cultured cells from at least two of the plurality of the cell culture bags of co-cultured cells in the cavity of the cell holder; and processing the co-cultured cells to form the synthetic tissue control.

Clause 2, the method of clause 1, further comprising applying an amount of dye to the cavity to identify the co-cultured cells deposited in the cavity during the processing of the co-cultured cells.

Clause 3, the method of clause 1 or 2, further comprising determining the amount of dye to apply to the cavity based on a number of the co-cultured cells deposited in the cavity.

Clause 4, the method of at least one of clauses 1-3, wherein forming the cavity in the cell holder comprises: inserting an object into the fluidly gel while the fluidly gel is solidifying, a portion of the object having a shape that defines the cavity; and removing the object once the fluidly gel has solidified to form the cavity.

Clause 5, the method of at least one of clauses 1-4, further comprising embedding the synthetic tissue control on a paraffin block to form a synthetic tissue micro array.

Clause 6, the method of at least one of clauses 1-5, further comprising: applying a filter material over the cavity; and transferring the cell holder onto a pathology cassette, wherein the pathology cassette is processed to form the synthetic tissue control.

Clause 7, a method to form a synthetic tissue control for use in determining presence of cancer, the method comprising: co-culturing a plurality of cell culture bags of cells comprising normal cells and cancer cells of a type of cancer based on at least one cell culturing factors, the at least one cell culturing factors comprising a type of the cancer cells being cultured, a ratio of the cancer cells to the normal cells being cultured, and seeding density of the normal cells and the cancer cells being cultured; depositing at least two cell culture bags of co-cultured cells of the plurality of cell culture bags of co-cultured cells in a cavity of a cell holder; processing co-cultured cells from the at least two cell culture bags of co-cultured cells deposited in the cavity; applying an amount of dye to the cavity to identify the co-cultured cells deposited in the cavity during the processing of the co-cultured cells; and forming the synthetic tissue control from the co-cultured cells deposited in the cavity.

Clause 8, the method of clause 7, wherein co-culturing the plurality of cell culture bags of cells comprises co-culturing the normal cells and the cancer cells in a cell culture chamber configured to maintain a concentration of CO₂ and temperature inside the cell culture chamber at a level based on the at least one cell culturing factors.

Clause 9, the method of clause 7 or 8, wherein co-culturing the plurality of cell culture bags of cells further comprises maintaining the cell culture chamber in a motorized rotating device operable to: hold the synthetic tissue control; and spin the cell culture chamber at a speed based on the at least one cell culturing factors.

Clause 10, the method of at least one of clauses 7-9, wherein co-culturing the plurality of cell culture bags of cells further comprises culturing the co-cultured cells to provide an expression of markers employed in IHC as diagnostic and as predictive markers for treatment response.

Clause 11, the method of at least one of clauses 7-9, wherein co-culturing the plurality of cell culture bags of cells further comprises culturing the co-cultured cells to provide an expression of markers employed in IHC as diagnostic markers or as predictive markers for treatment response.

Clause 12, the method of at least one of clauses 7-9, wherein co-culturing the plurality of cell culture bags of cells further comprises culturing the co-cultured cells to provide a consistent level of expression of RNA and DNA markers when observed via a FISH technique.

Clause 13, the method of at least one of clauses 7-9, wherein co-culturing the plurality of cell culture bags of cells further comprises culturing the co-cultured cells to provide a consistent level of expression of RNA and DNA markers when observed via a CISH technique.

Clause 14, the method of at least one of clauses 7-13, further comprising adding DNase to facilitate un-clumping of the normal cells and the cancer cells.

Clause 15, the method of at least one of clauses 7-14, further comprising: forming a homogenous core with un-clumped normal cells; and invading the homogenous core with un-clumped cells of the cancer cells.

Clause 16, the method of at least one of clauses 7-15, further comprising adding Fibronectin to facilitate un-clumping of the normal cells and the cancer cells.

Clause 17, a synthetic tissue microarray comprising: a plurality of synthetic tissue control, each synthetic tissue control of the plurality of synthetic tissue control comprising: normal cells; and cancer cells of a type of cancer, wherein the normal cells and the cancer cells are co-cultured in at least two cell culture bags of a plurality of cell culture bags of co-cultured normal cells and cancer cells, and wherein the plurality of cell culture bags of co-cultured normal cells and the cancer cells are co-cultured based on at least one cell culturing factors comprising a type of the cancer cells being cultured, a ratio of the cancer cells to the normal cells being cultured, seeding density of the normal cells and the cancer cells being cultured, and a type of cell growth supplement used to facilitate culturing the normal cells and cancer cells.

Clause 18, the synthetic tissue microarray of clause 17, wherein the normal cells and cancer cells are co-cultured to provide a high expression of markers used in IHC or ISH staining.

Clause 19, the synthetic tissue microarray of clause 17 or 18, wherein the normal cells and cancer cells are co-cultured to provide a medium expression of markers used in IHC or ISH staining.

Clause 20, the synthetic tissue microarray of at least one of clauses 17-19, wherein the normal cells and cancer cells are co-cultured to provide a low expression of markers used in IHC or ISH staining.

As used herein, an “approximately zero gravity environment” is defined to include a zero gravity environment.

As used herein, the singular forms “a”, “an” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and figures are merely illustrative and do not imply that any particular step or component is a requirement of a claimed embodiment.

What is claimed is:

1. A method to increase yield of co-cultured cells of a synthetic tissue control, the method comprising:
solidifying a fluidly gel form a solid cell holder;
forming a cavity in the cell holder to hold co-cultured cells, the cavity being operable to hold a plurality of cell culture bags of co-cultured cells, the co-cultured cells comprising normal cells and cancer cells of a type of cancer co-cultured based on at least one cell culturing factors, the at least one co-culture factors comprising a type of the cancer cells being co-cultured, a ratio of the cancer cells to the normal cells being co-cultured, seeding density of the normal cells and the cancer cells being co-cultured, a type of cell growth supplement used to facilitate co-culturing the normal cells and the cancer cells, and a concentration of the cell growth supplement used to facilitate co-culturing the normal cells and the cancer cells;
depositing co-cultured cells from at least two of the plurality of the cell culture bags of co-cultured cells in the cavity of the cell holder; and
processing the co-cultured cells to form the synthetic tissue control.

2. The method of claim 1, further comprising applying an amount of dye to the cavity to identify the co-cultured cells deposited in the cavity during the processing of the co-cultured cells.

3. The method of claim 2, further comprising determining the amount of dye to apply to the cavity based on a number of the co-cultured cells deposited in the cavity.

4. The method of claim 1, wherein forming the cavity in the cell holder comprises:
inserting an object into the fluidly gel while the fluidly gel is solidifying, a portion of the object having a shape that defines the cavity; and
removing the object once the fluidly gel has solidified to form the cavity.

5. The method of claim 1, further comprising embedding the synthetic tissue control on a paraffin block to form a synthetic tissue micro array.

6. The method of claim 1, further comprising:
applying a filter material over the cavity; and
transferring the cell holder onto a pathology cassette, wherein the pathology cassette is processed to form the synthetic tissue control.

7. A method to form a synthetic tissue control for use in determining presence of cancer, the method comprising:
co-culturing a plurality of cell culture bags of cells comprising normal cells and cancer cells of a type of cancer based on at least one cell culturing factors, the at least one cell culturing factors comprising a type of the cancer cells being cultured, a ratio of the cancer cells to the normal cells being cultured, and seeding density of the normal cells and the cancer cells being cultured;
depositing at least two cell culture bags of co-cultured cells of the plurality of cell culture bags of co-cultured cells in a cavity of a cell holder;
processing co-cultured cells from the at least two cell culture bags of co-cultured cells deposited in the cavity;
applying an amount of dye to the cavity to identify the co-cultured cells deposited in the cavity during the processing of the co-cultured cells; and
forming the synthetic tissue control from the co-cultured cells deposited in the cavity.

8. The method of claim 7, wherein co-culturing the plurality of cell culture bags of cells comprises co-culturing the normal cells and the cancer cells in a cell culture chamber configured to maintain a concentration of CO₂ and temperature inside the cell culture chamber at a level based on the at least one cell culturing factors.

9. The method of claim 8, wherein co-culturing the plurality of cell culture bags of cells further comprises maintaining the cell culture chamber in a motorized rotating device operable to:
hold the synthetic tissue control; and
spin the cell culture chamber at a speed based on the at least one cell culturing factors.

10. The method of claim 8, wherein co-culturing the plurality of cell culture bags of cells further comprises culturing the co-cultured cells to provide an expression of markers employed in IHC as diagnostic and as predictive markers for treatment response.

11. The method of claim 8, wherein co-culturing the plurality of cell culture bags of cells further comprises culturing the co-cultured cells to provide an expression of markers employed in IHC as diagnostic markers or as predictive markers for treatment response.

12. The method of claim 8, wherein co-culturing the plurality of cell culture bags of cells further comprises culturing the co-cultured cells to provide a consistent level of expression of RNA and DNA markers when observed via a FISH technique.

13. The method of claim 8, wherein co-culturing the plurality of cell culture bags of cells further comprises culturing the co-cultured cells to provide a consistent level of expression of RNA and DNA markers when observed via a CISH technique.

14. The method of claim 7, further comprising adding DNase to facilitate un-clumping of the normal cells and the cancer cells.

15. The method of claim 14, further comprising:
forming a homogenous core with un-clumped normal cells; and
invading the homogenous core with un-clumped cells of the cancer cells.

16. The method of claim 7, further comprising adding Fibronectin to facilitate un-clumping of the normal cells and the cancer cells.

17. A synthetic tissue microarray comprising:
a plurality of synthetic tissue control, each synthetic tissue control of the plurality of synthetic tissue control comprising:
normal cells; and
cancer cells of a type of cancer,
wherein the normal cells and the cancer cells are co-cultured in at least two cell culture bags of a plurality of cell culture bags of co-cultured normal cells and cancer cells, and
wherein the plurality of cell culture bags of co-cultured normal cells and the cancer cells are co-cultured based on at least one cell culturing factors comprising a type of the cancer cells being cultured, a ratio of the cancer cells to the normal cells being cultured, seeding density of the normal cells and the cancer cells being cultured, and a type of cell growth supplement used to facilitate culturing the normal cells and cancer cells.

18. The synthetic tissue microarray of claim 17, wherein the normal cells and cancer cells are co-cultured to provide a high expression of markers used in IHC or ISH staining.

19. The synthetic tissue microarray of claim 17, wherein the normal cells and cancer cells are co-cultured to provide a medium expression of markers used in IHC or ISH staining.

20. The synthetic tissue microarray of claim 17, wherein the normal cells and cancer cells are co-cultured to provide a low expression of markers used in IHC or ISH staining.
